(19) United States
(12) Patent Application Publication
(10) Pub. No.: US 2021/0062180 A1
(43) Pub. Date: Mar. 4, 2021

(54) SEMI-AUTOMATED RESEARCH INSTRUMENT SYSTEM

(71) Applicant: Sage Science, Inc., Beverly, MA (US)

(72) Inventors: Ezra ABRAMS, Newton, MA (US); Todd J. BARBERA, Marblehead, MA (US); Chris BOLES, Bedford, MA (US)

(21) Appl. No.: 16/959,989

(22) PCT Filed: Jan. 4, 2019
(86) PCT No.: PCT/US2019/012416

§ 371 Date: Jul. 2, 2020

Related U.S. Application Data
(60) Provisional application No. 62/614,239, filed on Jan. 5, 2018.

(51) Int. Cl. C12N 15/10; G01N 27/447; B01L 3/00

(52) U.S. Cl. CPC C12N 15/101; C12N 15/1017; B01L 2300/0816; B01L 3/502715; G01N 27/44704

(57) ABSTRACT

A cassette for retaining molecules during electrophoresis has a housing with a lane configured therein. The lane has a first elongate edge and a second elongate edge, and an elution module is configured to be received in the late to divide the lane into a first chamber and a second chamber. A first buffer reservoir is positioned adjacent the first elongate edge, and a second buffer reservoir is positioned adjacent the second elongate edge. The first side of the elution module facing the first chamber comprises a porous sterile filtration membrane. The second side of the elution module facing the second chamber comprises an ultrafiltration membrane that has a pore size to retain molecules during electrophoresis.

### CROSS-REFERENCE TO RELATED APPLICATIONS

This application claims priority to and the benefit of U.S. Provisional Application No. 62/614,239, filed Jan. 5, 2018, and entitled, “A Semi-Automated Research Instrument System.” The present application expressly herein incorporates by reference the disclosure of the above-referenced application in its entirety.

### BACKGROUND

Next-generation sequencing (NGS) is increasingly being used m medicine for diagnosis of inherited genetic disease and for selection of appropriate therapies in oncology, Since many inherited disease and actionable cancer loci are single nucleotide polymorphisms (SNPs) within protein-coding sequences, most clinical sequencing (i.e., the Illumina and Ion Torrent platforms) is carried out using short-read sequencing in combination with hybridization-capture targeted sample preparation (Agilent SureSelect, Roche Nimblegen, IDT xGen).

However, short-read NOS methods are not well suited to long-range genomic analyses, such as haplotype determination and detection of structural variation (“SV”), defined here as rearrangements involving deletions, duplications, inversions, translocations. repeat expansions, greater than 100 bp in length. Short-read methods are particularly disadvantaged when SV break points and haplotypes involve regions of repeated sequence.

This presents a significant problem for the clinical sequencing field since recent studies on large patient cohorts suggest that some types of cancer contain driver mutations caused by SVs rather than by SNPs (4). Another population study of cancer (Pan-Cancer Analysis of Whole Genomes) found significantly recurrent SVs at 52 different loci (10). Similarly, there is growing awareness that a significant (but yet unknown) fraction of inherited disease is caused by deletions, repeat expansions, and other SVs which cannot be detected by short read methods (11, 12). Finally, there is accumulating evidence that haplotype structures extending across the entire 4 mb MHC region will be important for deciphering the many complex immune disorders (reviewed in 13). Here again, short read methods can identity discrete points of polymorphism, but linkage between therm can only be inferred (indirectly) from analyses of families.

Reliable detection of SVs and extended haplotypes require long-read single molecule sequencing (or optical mapping methods) such as those developed by Pacific Biosciences, Oxford Nanopore, 10× Genomics, Bionanogenomics, and Genomic Vision (2-12). As in the case of short read gene panels and exome sequencing, it is likely that use of long-read sequencing in clinical settings will be limited to targeted sequencing, for economic reasons. Unfortunately, targeted sample preparation for these long-read sequencing methods remains laborious, inefficient, and expensive compared with conventional targeted short-read sequencing.

### SUMMARY OF SOME OF THE EMBODIMENTS

Various apparatuses, systems, and methods are described herein. In some embodiments, a molecule retention cassette for retaining molecules during electrophoresis includes a housing and a lane configured within the housing. The lane may have a first elongate edge arid a second elongate edge. An elution module may be configured to be received in the lane and may divide the lane into a first chamber and a second chamber. A first buffer reservoir may be positioned adjacent the first elongate edge, and a second buffer reservoir may be positioned adjacent the second elongate edge. A first side of the elution module facing the first chamber may comprise a porous sterile filtration membrane, and a second side of the elution module facing the second chamber may comprise an ultrafiltration membrane that has a pore size to retain molecules during electrophoresis.

In some embodiments, the cassette may further comprise at least one electrode configured within the first chamber and at least one electrode configured within the second chamber.

The ultrafiltration membrane may be a 15 kDa ultrafilter. The pore size of the ultrafiltration membrane may be configured to retain DNA during electrophoresis.

The elution module may be centrally positioned between the first buffer reservoir and the second buffer reservoir. The elution module may be positioned between the first buffer reservoir and the second buffer reservoir. In some embodiments, the elution module may comprise the elution module and the sample well. The elution module may be configured to receive a sample.

The cassette may further comprise agarose gel. The agarose gel may be cast next to the porous sterile filtration membrane. The agarose gel may be cast to form a gel column, and dimensions of the gel column are configured to minimize loss of target molecules into the first chamber.

In some embodiments, a molecule retention cassette for retaining molecules during electrophoresis comprises a housing and a plurality of lanes configured within the housing. The plurality of lanes each has a first elongate edge and a second elongate edge. A plurality of elution modules may each be configured to be received in a lane of the plurality of lanes so as to divide each lane into a first chamber and a second chamber. A first buffer reservoir may be positioned adjacent the first elongate edge of each lane, and a second buffet reservoir may he positioned adjacent the second elongate edge of each lane. A first side of the elution module facing the first chamber of each lane comprises a porous sterile filtration membrane, and a second side of the elution module facing the second chamber of each lane comprises an ultrafiltration membrane. The ultrafiltration membrane has a pore size to retain molecules during electrophoresis.

In some embodiments, a method for isolating and collecting target segments of target particles may include receiving a sample in a sample well of an elution module. An SDS-containing lysis buffer may be received in a first buffer chamber, the first buffer chamber being configured along a first side of the elution module, A first electrophoresis voltage may be applied to migrate components of the sample towards a second buffer chamber that is configured along a second side of the elution module, surer that target particles are immobilized in a gel segment configured along the second side of the elution module between the elution module and the second buffer chamber, and non-target particles pass through the gel segment and into the second buffer chamber. The first buffer chamber, the second buffer chamber, and the elution module may be washed and filled with a Cas9 reaction buffer. The elution module may be emptied and refilled with a Cas9 enzyme mix to cleave sections of the target particles immobilized in the gel segment, An SDS stop solution may be loaded into the elution, module, and a second electrophoresis voltage may be applied to release the Cas9 from the target particles and migrate Cas9 into the second buffer chamber. The first buffer chamber, the second buffer chamber, and the elution module may be washed and filled with elution buffer. A third electrophoresis voltage may be applied in a reverse direction to migrate the cleaved sections of the target particles from the gel segment and into the elution module.

The first side of the elution module may comprise an ultrafilter and the second side of the elution module, may comprise a porous sterile filter. The porous sterile filter may prevent the cleaved sections of the target particles from leaving the elution module during and after the application of the third electrophoresis voltage.

The target particles may be DNA and the cleaved sections of the DNA may comprise desired genomic targets.

In some embodiments, application of the second electrophoresis voltage may he shorter than the first electrophoresis voltage and/or the third electrophoresis voltage.

Application of the first electrophoresis voltage may migrate SDS through the elution module to lyse the sample and coat the non-target particles of the sample such that the non-target particles pass through the gel segment and into the second buffer chamber. Application of the second electrophoresis voltage migrates particles smaller than the target particles into the second buffer chamber.

An electrophoretic instrument system may include an electrophoresis station and a drawer configured to receive at least one electrophoresis cassette, such as those described herein. The system may also comprise a liquid handling robot and a lateral extension arm that is configured to move the drawer laterally, such that moving the drawer in a first lateral direction exposes a first side of the at least one cassette to the liquid handling robot, and moving the drawer in a second lateral direction inserts the drawer into the electrophoresis station. The electrophoresis section houses electrodes that correspond to the at least one cassette, wherein the electrodes are configured to apply electrophoresis voltages. The system may also include at least one cold storage compartment and at least one room temperature storage compartment.

### BRIEF DESCRIPTION OF THE DRAWINGS

FIG. 1A shows a SageHLS cassette, according to some embodiments.

FIG. 1B shows a SageHLS instrument, according to some embodiments.

FIG. 2 shows an HLS-CATCH workflow for isolation of large genomic DNA targets, according to some embodiments.

FIG. 3 shows targeted 10× Genomics sequencing of a 200 kb BRCA1 fragment prepared by HLS-CATCH, according to some embodiments.

FIG. 4 shows fully phased output of BRCA1 sequence from FIG. 3 after processing by 10× Long Ranger software, according to some embodiments.

FIG. 5 shows SV detection on HIS-CATCH targets using 10× Genomics/Illumina sequencing, according to some embodiments.

FIG. 6 shows an initial concept for compact multichannel CATCH-1D cassette prototype, according to some embodiments.

FIG. 7 shows a CATCH-1D workflow, according to sonic embodiments,

FIG. 8 shows an exemplary flow chart, according to sonic embodiments.

FIG. 9 shows an example of a possible liquid handler deck layout of an automated CATCH-1D instrument, illustrating integration of an electrophoresis station onto the deck of a liquid handling robot, where the dimensions refer to the footprint of a typical small OEM liquid handling station, according to some embodiments.

### DETAILED DESCRIPTION OF SOME OF THE EMBODIMENTS

An integrated, high-throughput, automated sample prep system for targeted long-read sequencing is developed. The proposed system is intended for robust walk-away automated processing in a clinical diagnostic setting.

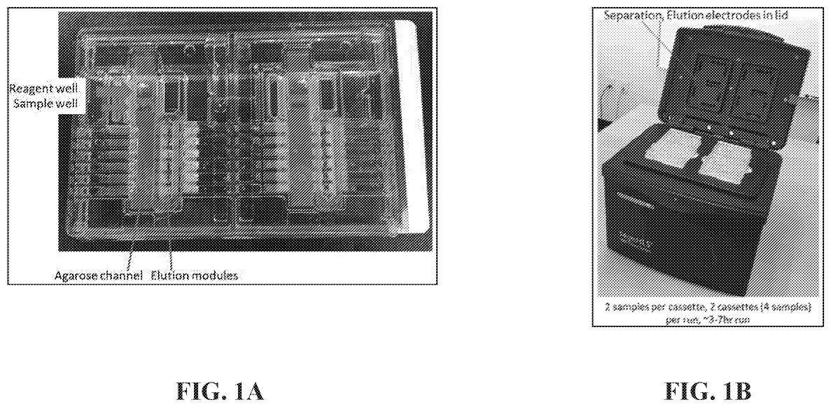

A semi-automated research instrument system (FIGS. 1A-B) is provided for extraction and enzymatic processing of extremely high molecular weight (HMW) DNA (100-2000 kb). The system uses intact cells or isolated nuclei as input samples. Input samples are loaded into an agarose gel cassettes (FIG. 1A), and chromosome length DNA is extracted from the samples by electrophoresis of SDS through the sample well compartment. SDS-coated proteins, lipids are electrophoresed away from the sample well through the central agarose gel column, but the chromosome-length DNA becomes firmly entangled and immobilized in the agarose gel wall of the sample well. The sample well can be emptied and refilled without any loss of DNA. This allows for treatment the immobilized DNA by refilling the sample well with an enzyme reaction mixture. Many commonly used DNA processing enzymes readily diffuse into the agarose, including many restriction enzymes, DNA polymerases, ligases, transposases, non-specific DNA cleavases, and S. pyogenes Cas9. After DNA processing, an additional round of size-selection electrophoresis is performed, followed by electroelution of the DNA products into a series of six buffer-filled elution modules arranged along one side of the gel separation column (FIG. 1A). The DNA processing step includes some cleavage to reduce the size of the desired DNA products to below 2 megabases (mb) in length DNA greater than 2 mb will remain immobilized in the sample well, unable to move during electrophoresis.

As shown in FIG. 1A, each cassette has two physically isolated sample processing areas. The cassette has a standard 96-well plate footprint. The central agarose channel has two loading wells. Cells or nuclei are loaded in the sample well, and art SDS-based lysis reagent is loaded into the reagent well. Electrophoresis is carried out to drive the SDS through the sample well compartment where the cells or nuclei are lysed. Chromosome-sized genomic DNA becomes immobilized in the sample well wall, while other components are carried to the bottom electrode chamber along with the SDS. After DNA processing and size selection electrophoresis, the DNA products are electrocluted into an array of six elution modules positioned along the right side of the agarose channel.

FIG. 1B shows the instrument is approx. 1 ft wide/tall/deep and holds two cassettes. Total sample throughput is 4 samples, and nm times vary between 3 and 7 hours depending on the size range and desired resolution of the size selection step.

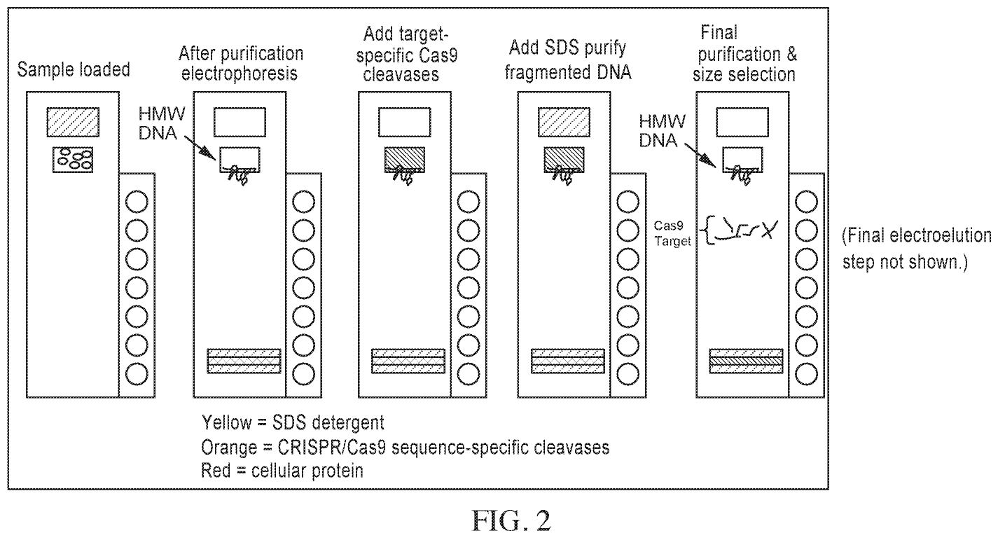

The SageHLS system can be used with customized Cas9 nucleases to isolate specific large genomic DNA targets that can be efficiently used to prepare targeted DNA libraries in the 10× Genomic Chromium system. FIG. 2 shows a schematic of the workflow, referred to herein as “CATCH.” Intact cells at nuclei arc loaded into the cassettes and electrophoretic extraction is carried out as described above, leaving the chromosomal-length genomic DNA immobilized in the sample well wall. Enzymatic DNA processing is carried out using customized Cas9 cleavases which are designed to cleave outside oldie genomic region (or regions) to be sequenced. After Cas9 digestion, size selection electrophoresis is carried out to move the Cas9-cleaved target regions away from the sample well and away from the uncleaved genomic DNA, which remains trapped in the sample well wall. After electroelution from the gel channel, the products are localized by qPCR, arid used directly in the 10× Chromium system for library construction.

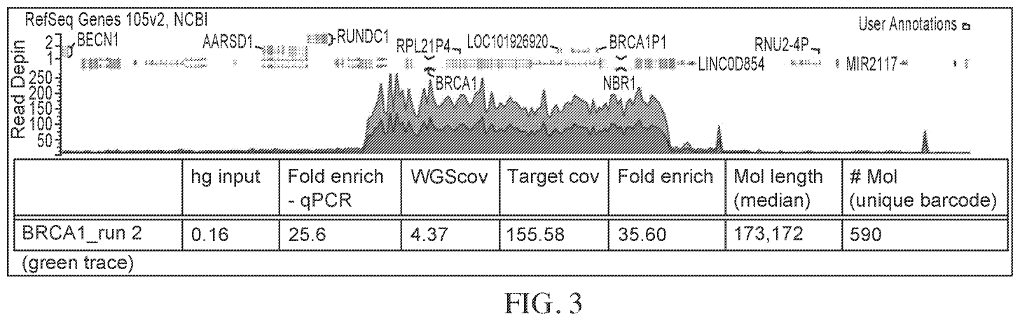

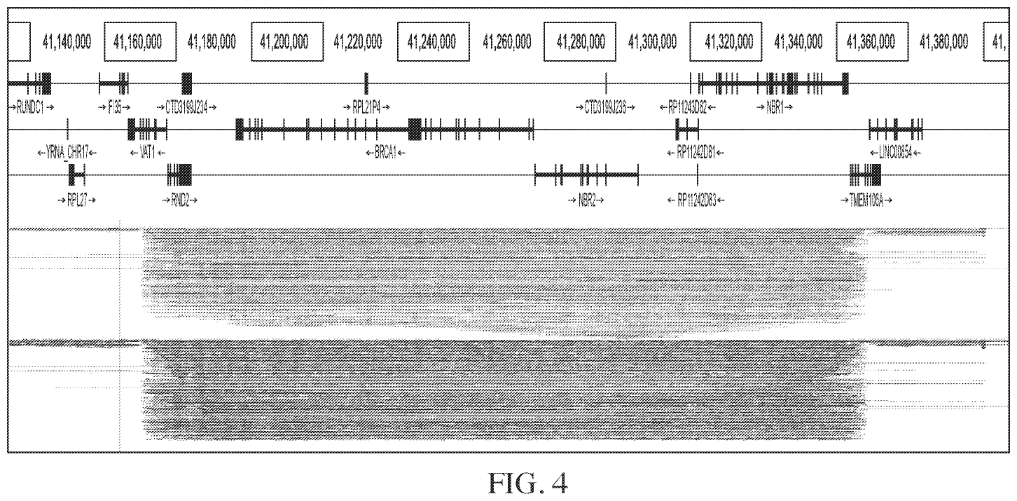

The overall workflow for targeted sequencing of a 200 kb genomic region encompassing the human BRCA1 gene has been demonstrated. Using 1.5 million human diploid cultured cells as the input material, approximately 30,000-50,000 copies of the BRCA1 Fragment were recovered from the peak elution module, with an enrichment of ˜25-fold over a non-targeted control gene (RNaseP) as measured by Taqman qPCR. After preparation of libraries on the 10× Genomics Chromium system, they were sequenced on an Illumina NextSeq500 system. The run produced 155× coverage of the targeted BRCA1 region, with only 4.4× coverage of the non-targeted remainder of the genome (FIG. 3). Further analysis using the 10× Long Ranger alignment software, demonstrated that fully phased haplotypes could be determined for both alleles (FIG. 4). More than half of the BRCA1 HLS-CATCH products were full-length targets as predicted from the gRNA designs.

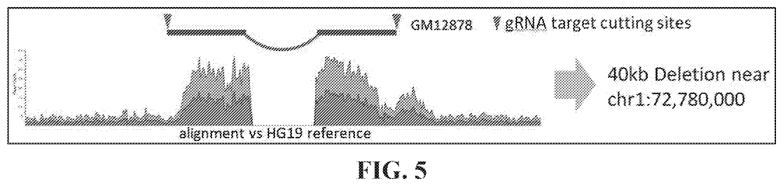

In parallel experiments, it has been demonstrated that HLS-CATCH targeting can also be used to detect large SVs, as gRNAs targeting 40 known SVs in the well-studied cell line GM12878 were designed. The targeted regions were isolated in a single multiplex CATCH procedure targeting 40 distinct 100 kb genomic fragments. Targets were localized in the HLS cassette output by qPCR, and subjected to 10× Genomics library prep and Illumina sequencing as for the BRCA1 gene. Some preliminary data demonstrating detection of a homozygous 40 kb deletion on GM12878 chr1 is shown in FIG. 5 (Shin, Ji, manuscript in preparation).

The data of FIGS. 3-5 show that HLS-CATCH in combination with 10× Genomics library construction methods can provide high-quality long-range haplotype and SV calls in a targeted fashion. We believe that this is the first successful demonstration of a targeted sequencing workflow using genomic ligaments larger than 20 kb (14). The success of the combined technologies comes from the excellent complementarity between the efficient target enrichment of the HLS-CATCH process, and the integrated amplification+library construction process of the 10× Chromium workflow.

The diagnostic tests based on the HLS-CATCH/10× Genomics workflow outlined above may have compelling advantages in genetic testing and oncology. The targeted nature of the workflow offers substantial decreases in Illumina sequencing costs ($8000 in ILMN reagents for 100× phased Chromium whole genome sequence vs $500 in ILMN reagents for the 100× phased targeted Chromium coverage of BRCA1 in FIG. 4). In addition, analysis of large targeted fragments offers the chance to detect both SVs and SNPs in the same assay and with the added benefit of haplotype determination.

### Approach

it may be advantageous for systems used for such applications to have high sample throughput, such that the number of samples processed is increased and/or maximized. Additionally, it may be advantageous to achieve efficient, specific Cas9 target cleavage in a reproducible fashion. In approximately 30 HLS-CATCH experiments with mouse WBCs, and human cultured cells (lymphoblastoid and HEK293 cells), CATCH target recovery has varied widely, ranging between 2 and 50%. Similarly, target enrichment has varied between 15 and 20-fold. Fortunately, the 10× Chromium library prep works quite well with samples at the low ends of these ranges. For instance, the high-coverage targeted data of FIGS. 3-5 were produced with an overall CATCH target recovery of only about 1.5% (50,000 copies), and enrichment of about 25-fold (both values measured by qPCR relative to RNaseP gene copy number). This may be another complementary feature of the HLS-CATCH/10× Genomics workflow: the success of the overall workflow depends on substantial enrichment, but not on absolute target purity, which in the latter case would lead to inefficiencies due to “bar-code collision” where multiple alleles would co-locate in droplets and be labeled with the same linked-read barcodes.

It may also be advantageous to modify and extend HLS-CATCH to work with very low cell/nuclei inputs This is a key issue for genotyping diagnostic samples such as biopsy samples, which may be very limited in size and cell number. Success in using low cell/nuclei inputs will depend on the success in optimizing the Cas9 digestion conditions, as discussed above.

Furthermore, it may be important to fully evaluate and extend the capabilities of the HLS-CATCH for using a variety of tissue types, including buffy coat, frozen blood, fresh and fresh frozen solid tissues including tumor biopsy materials of various types,

In Phase I, a cassette may be designed to accomplish HLS-CATCH large target enrichment. The new design may eliminate the two-dimensional electroelution step of the original HLS cassette, and thereby allow manufacture of cassettes capable of processing between 6 and 12 samples per cassette (in a 96 well plate footprint) The new cassette type is referred to herein as “CATCH-1D” where ID stands for “one-dimensional.”

Phase I may include using a sample of 750,000 diploid mammalian cells (human or mouse) to achieve a recovery of at least 20% (300,000 copies) of a single-copy 200,000 bp genomic target fragment in the CATCH-1D prototype. The copy number may be measured by qPCR. The enrichment of the 200,000 bp genomic target may be at least 20-fold over non-targeted gnomic sequence background, and enrichment may be measured by qPCR. Additionally, it may be demonstrated that the CATCH-1D prototype produces CATCH targets that can be sequenced efficiently in the 10× Genomics Chromium, Illumina workflow.

Phase II may include development of a CATCH-1D instrument with liquid handling capabilities for full high-throughput walk-away automated CATCH target preparation. Cas9 digestion may be optimized far CATCH-1D operation, Phase II may also include adapting the CATCH-1D cassette or workflows for use with low cell or nuclei input and adapting CATCH-1D cassette or workflows for use with diagnostically i:important tissue types.

### Phase I Approach

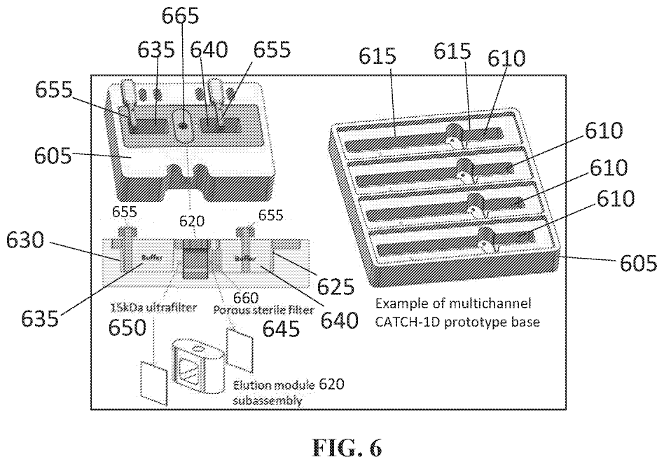

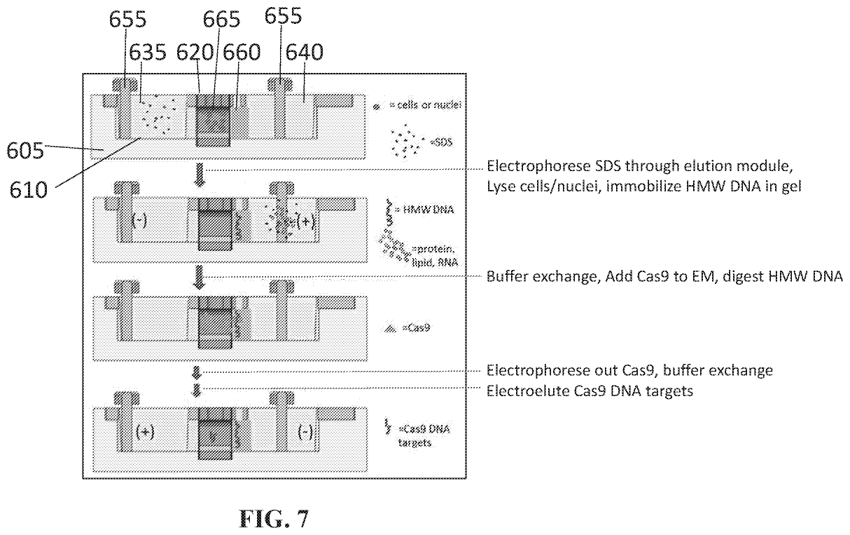

The basic concept of the CATCH-1D cassette and its operation is shown in FIGS. 6 and 7. The SageHLS cassette (FIG. 1A) is modified so that each lane 610 of the housing 605 comprises a pair of buffer reservoirs 635, 640 on either side am elution module 620. Each lane 610 has an elongate edge 615 on either side of a slot configured to receive an elution module 615. When the elution module 620 is inserted into the corresponding slot, the lane is divided into a first chamber 625 and second chamber 630. The first chamber 625 may comprise a first buffer reservoir 635 and the second chamber 630 may comprise a second buffer reservoir 640,

In some embodiments, the elution module 620 is centrally positioned, approximately centrally positioned, or otherwise situated between the buffer reservoirs 635, 640, On one side of the elution module 620, a porous sterile filtration membrane 645 is attached. A small segment of agarose gel 660 is cast against the outside surface of the sterile filter 645. On the other side of the elution module 620, an ultrafiltration membrane 650 is attached. The ultrafilter 650 has a pore size which will retain DNA during electrophoresis.

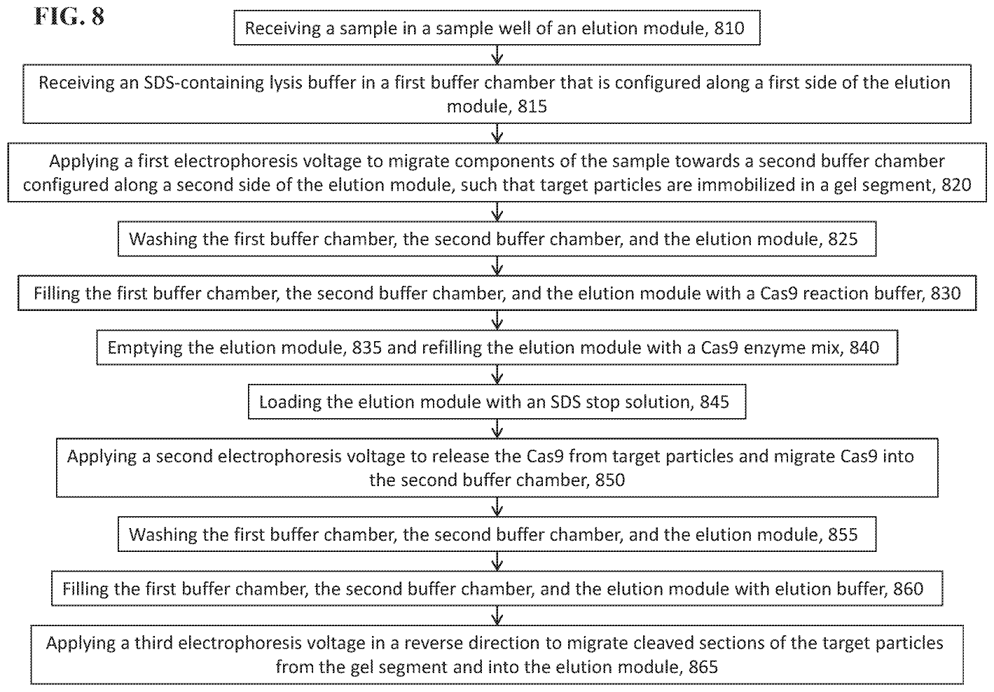

In the CATCH-1D workflow, the elution module 620 serves both as the sample well 665 and the elution module 620. As illustrated in FIG. 7 and shown in the flow chart of FIG. 8, the cell/nuclei sample is loaded into the elution module 620, 810, and the left buffer chamber 635 is emptied and refilled with all SDS-containing lysis buffer 815. Electrophoresis voltage is applied 820, e.g., via one or more electrodes 655, so that SDS migrates through the elution module, lysing the input material, and coaling proteins and other non-DNA cellular components so that they migrate through the small gel segment 660 and into the right buffer chamber 640. During this period, the genomic DNA migrates into the small gel segment 660 and becomes immobilized there, as in the original HLS process (original process shown in FIG. 2). At this point, electrophoresis is discontinued and ail three compartments of the cassette (left and right buffer chambers 635, 640, and elution module 620) are washed thoroughly 825, and refilled with Cast) reaction buffer 830. The elution module 620 is then emptied 835 and refilled with Cas9 enzyme mix 840 which will specifically cleave the desired genomic targets from the chromosome-length genomic DNA which is immobilized in the gel 660. After digestion, the elution module 620 is loaded with an SDS stop solution 845, and electrophoresis is carried out 850 so that Cas9 released from the DNA and moved into the right buffer chamber 640. The electrophoresis period required for this cleanup is very brief because the denatured Cas9 protein will migrate much faster than the large DNA CATCH targets in the agarose gel 660. After this Cas9 cleanup electrophoresis, the cassette is washed 855 and refilled with elution buffer 860. Electrophoresis is carried out in the reverse direction 865 to move the CATCH products from the small gel segment 660 into the elution module 620, where the ultrafiltration membrane 650 on the left side of the elution module 620 prevents the targets from escape.

The design of the CATCH-1D cassette is much simpler than the original SageHLS cassette, and fabrication of the prototype can be performed. A Sage Science PippinHT instrument (such as that shown in Appendix A) with customized electrode arrays for CATCH-1D prototype testing can be used. The PippinHT instrument can be modified to accept cassettes with up to 12 channels. All liquid handling steps may be performed manually (e.g., during Phase I) or may be automated (e.g, during Phase II).

There arc several reagent contamination risks in the CATCH-1D workflow which are different from the original HLS workflow. One example comes from using the elution module as the input sample well. Some components of the input sample may adhere to the elution module surfaces and prove hard to remove during lysis and washing steps, there is some risk that some residual amount of SDS may remain in the elution module following initial lysis or Cas9 cleanup steps. (The elution modules of the original HLS cassette are not exposed to input sample or concentrated SDS).

Another possible challenge is adjusting the gel column dimensions so that electrophoretic Cas9 cleanup with SDS can be accomplished without loss of CATCH target into the right buffer reservoir. It is not anticipated that this will be a serious challenge for CATCH targets greater than 100 kb, and target loss by qPCR of the right chamber buffer (after Cas9 cleanup electrophoresis) can be measured.

Another significant risk to the CATCH-1D workflow is the absence of a size-selection electrophoresis step. In the original HLS-CATCH workflow, a size-selection electrophoresis is performed to increase the purity of the CATCH product(s) just before elution. The proposed CATCH-1D workflow omits size selection electrophoresis, although the electrophoretic Cas9 cleanup step offers the possibility to remove low molecular weight DNA that migrates significantly faster than the CATCH target DNA, In any case, it is likely some non-specifically cleaved HMW DNA will be dined along with the CATCH products in the final elution, and purity of the CATCH products will not be as high as in the HLS-CATCH workflow, This may not be problematic, however, as excellent 10× sequencing results can be obtained with modest enrichment factors (˜15-25-fold). In addition, further optimization of Cas9 digestion conditions, better gRNA design, and new mutant Cas9 enzymes (or similar programmable endonucleases) could reduce the importance of size-selection.

### Phase II Approach

In Phase I, feasibility of the CATCH-1D system is demonstrated. Phase II includes development of the fully automated high-throughput CATCH-1D system.

### CATCH-1D Cassette and Instrument Development

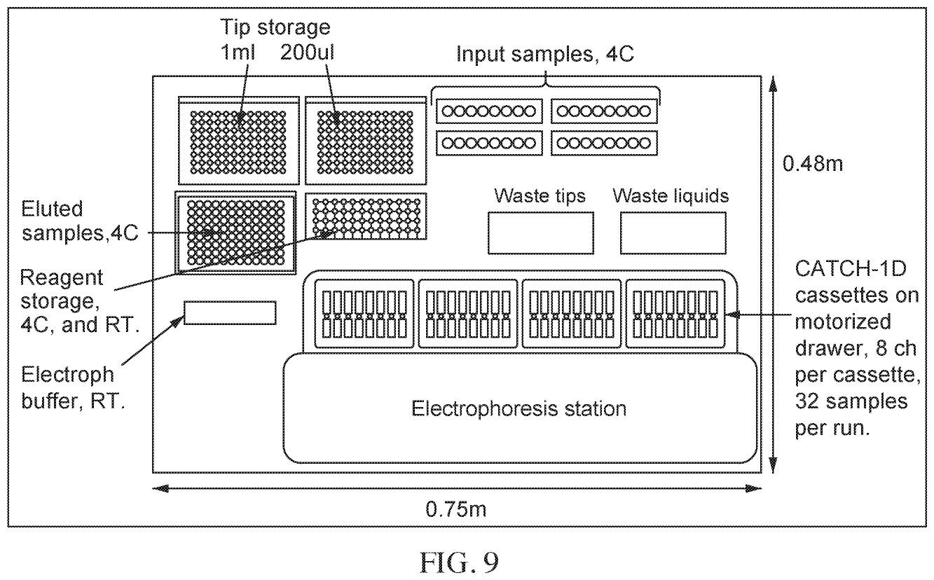

Phase II features a detailed systems engineering study on integrating of liquid handling functions with electrophoresis in an automated CATCH-1D system. An exemplary automated instrument is illustrated in FIG. 9. An clectrophoresis station is integrated directly onto the deck of a small OEM gantry-style liquid handling robot. The electrophoresis station may have a motorized drawer, which may hold four CATCH-1D multichannel cassettes. The drawer can be extended laterally to expose the tops of the cassettes for liquid handling steps (loading samples/reagents, cassette washing, removing products), and withdrawn inside the station (which houses the electrodes) for the electrophoresis steps. A single liquid handling head may be sufficient, with the number of LH channels matching the number of channels per cassette (in FIG. 9, we have shown eight channels per cassette). As shown in FIG. 9, accommodations for cold (4C) storage may be provided for input samples and Cas9 reagents, RT storage for the lysis reagent and electrophoresis buffer, storage for disposable tips, and waste chambers for used tips and waste liquids.

A prototype multichannel CATCH-1D cassette may be fabricated, which may include determining an optimum number of samples processed per cassette and the sample input size. Additionally, the efficiency of DNA extraction and Cas9 digestion as a function of channel/elution module dimensions (as explained below) may be determined. The channel of the CATCH-1D cassette may be small, so that many samples can be processed in each cassette. A determination may also be made to ensure that enough CATCH target is extracted to get ample 10× Genomics linked-read coverage for diagnostic utility.

After evaluating channel dimension effects on extraction and CATCH recovery, a second, substantially optimized CATCH-1D cassette may also be built. An initial instrument prototype that can perform a fully automated test with the version 2 cassette may also be built. Production versions of the CATCH-1D cassette and automated instrument may also be developed. Manufacturing protocols may be determined for the final production versions of cassette and instrument, and the final system may be tested.

Optimization of HMW DNA extraction in CATCH-1D

The overall efficiency of the CATCH-1D process is a composite of two values. The first is the efficiency of the initial genomic DNA extraction from the input sample. The second is the efficiency of recovery of Cas9-digested CATCH targets. It is believed that the efficiency of the initial genomic DNA extraction in CATCH-1D is a function of: 1) the surface area of the agarose gel sample well where cell lysis arid DNA immobilization occurs (larger SA is better), and 2) the amount of detergent used per cell during extraction (more is better). To examine this issue, multichannel CATCH-1D prototypes may be fabricated with different agarose gel cross-sectional areas, and extraction efficiency may be evaluated as a function of sample input. Extraction efficiency may be measured by total DNA recovery after digesting the immobilized DNA with a frequent cutting restriction enzyme and electrocuting the digest. Extraction efficiency for long genomic DNA fragments (100-200 kb) may be measured by digesting the immobilized DNA with rare-cutting restriction enzymes and testing the electrocuted yield of specific genomic DNA restriction fragments of known length. Recovery of specific genomic DNA fragments may be measured by qPCR. The maximum input load evaluated may be 750,000 diploid human cells, since phase I work may demonstrate that that input will produce ample CATCH target (at least 300,000 copies) for high coverage in the 10× Chromium sequencing workflow.

### Optimization of Cas9 Digestion and CATCH Target Recovery

Once extraction efficiency scales with channel/elution module cross-section are understood, studies on the efficiency of Cas9 digestion may be initiated to determine how it scales with input sample load and channel dimensions, Since many kinds of clinical samples will have a low number of input cells, performance of the cassette prototypes may be evaluated and optimized at low cell inputs. It is possible to get good 10× Genomics sequence coverage (˜100×) in single target CATCH experiments with as few as 50,000 targets recovered. In several experiments, we have achieved a 50% recovery of a Cas9 target (the 200 kb BRCA1 locus target). At this recovery value, an input of only 50,000 intact cells (or nuclei) would be sufficient, for equivalent (˜100×) Chromium library sequencing coverage. When higher target recoveries are achieved, the required cell input would be lowered proportionally, which may be significant because many clinically relevant samples will have very low cell inputs. With human cell inputs in the low 10,000's, 100× targeted sequencing coverage may be achieved.

addition to optimizing the proper cassette channel dimensions for efficient extraction and CATCH target recovery gRNA design, gRNA type, Cas9 enzyme choice, and in-cassette Cas9 reaction conditions may also be addressed.

Cas9 reaction conditions (enzyme cone, and buffer) are the dominant factors for CATCH target recovery gRNA type, gRNA design, and Cas9 type have less effect, Almost every gRNA tested works to some degree on its intended target, and 75% of the guides show complete digestion of PCR products at equimolar target/Cas9 ratios (cone's in the 0.1-0.4 nM range). Moreover, a number of different gRNA design tools (guidescan.com, Feng Zhang lab website at MET, SureDesign tool from Agilent) may be used successfully. Furthermore, similar cutting efficiency may be seen using two-part synthetic, gRNAs and T7pol in vitro-transcribed single gRNAs (although we feel the synthetic RNAs are easier and more reliable to use). Commercially available Cas9 mutants have not been found to be more specific than wt-Cas9 enzyme in our in vitro assays.

Despite these initial conclusions, the CRISPR nuclease family is constantly expanding, and new mutants may be engineered that may be useful for our process. Thus, more enzymes for enhanced performance in the CATCH-1D cassette may be screened. Similarly, full-length synthetic single guide RNAs which are recently becoming commercially available may also be tested, The single guides may simplify the Cas9 assembly workflow. For example, Synthego may be used, as the supplier has advertised that their synthetic single guides have significantly improved specificity versus in vitro transcribed single gRNAs.

In the HLS-CATCH process, the best target recoveries have been achieved using 2-3 gRNAs per cut site window (multiple guides per cut site are used to avoid the possibility that a SNP in a single gRNA recognition sequence will eliminate a cut site), a cut site window of around 2 kb, and a total concentration of 1-4 uM Cas9 enzyme in the sample well (assembled at 1:1 ratio of Cas9:total gRNA concentration). In the SageHLS process, target recovery is also significantly enhanced by electrophoretically “injecting” the Cas9 enzyme into the wall of the sample well where the HMW DNA is immobilized. A 1 min period electrophoresis at ˜50V may be used. Enzyme concentration and electrophoretic injection are the two parameters that have the largest effect on CATCH target yield.

### Development of CATCH-1D Methods for Other Clinical Sample Types

The HLS workflows may be adapted for a variety of materials including whole blood, frozen whole blood, bully coat, fresh solid tissues, fresh frozen tissue, tumor biopsies, and nuclei obtained from all of the above sources. For the CATCH process, it may be critical that the extracted DNA is nearly full chromosome length, or >>2 mb in size, for efficient immobilization in the agarose gel to occur. This means that the CATCH process may work best with fresh material, or nuclei prepared from fresh material. It also means that CATCH processes are unlikely to work on formalin-fixed tissues.

SageHLS development was accomplished with mammalian WBCs or human cell lines, or isolated nuclei from those sources. The SageHLS system has been used to isolate HMW DNA, from ˜50 ul of whole blood, which should contain around 2.5 ug of genomic DNA, which is sufficient input for the CATCH process. In addition, SageHLS has successfully been used with frozen human tissue culture cells (HEK293) for isolation of HMW DNA. Given these two pieces of data, protocols for processing buffy coat, frozen buffY coat, and frozen whole blood may be found.

For tissue work, there have been several instrument+reagent systems for tissue dissociation developed by suppliers of cell cytometers and FACS instruments (BD Biosciences and Miltenyi Biotech). These involve a combination of mechanical and enzymatic treatments followed by selective filtration to produce suspensions of single cells or nuclei. These systems may perform well for CATCH-1D input with fresh materials.

Based on limited success with frozen tissue culture cells, the most viable clinical tissue sample for CATCH will be fresh-frozen tissue, Efforts may be focused around that sample type, and studies using the BD and Miltenyi instrumentation systems cited above may be initiated,

### Consistent Reproducible Workflow for Development of CATCH-1D Reagents Kits

The combination CATCH-1D+10× Genomics workflow may have bread applications in many areas of clinical sequencing. For this reason, development and optimization of the CATCH-1D system includes establishing optimal development paths for rapid production of new CATCH reagent kits. This may include integration of bioinformatics for genomic cut site selection, gRNA selection within cut site windows, streamlined pipeline for gRNA ordering and QC, and trackable product numbering, stocking, and packaging.

Clinical sequencing markets may be surveyed to select key diagnostic areas which may benefit from the CATCH-1D approach. For example, CATCH-1D may be used to enable long-range sequencing in the MHC region. Additionally, there arc other attractive assay areas in inherited disease testing and oncology.

An optimized workflow for design of gRNAs that can be applied to create CATCH assays for any region (or set of regions) of the human genome may be provided. Some assays in inherited disease testing, may only involve a single gene along with flanking regions. Other tests may involve a panel of cancer genes (perhaps low 100's) frequently involved in SVs (10). Still others may involve design of assays producing CATCH products that tile across a broad genomic region like the MHC (14).

Modifications, customization, or new versions of 10× Cienoinie Long Ranger software designed specifically for analysis of targeted CATCH-1D-F-10× Chromium sequencing results may be assessed.

A 10× Chromium sequencing workflow and QC method for validating performance of new kits may be developed. While qPCR is an inexpensive a fast method for assessing recovery and enrichment, a conical sequencing method may be developed.

Any and all references o publications or other documents, including but not limited to, patents, patent applications, articles, webpages, books, etc presented in the present application, are herein incorporated by reference in their entirety.

Example embodiments of the devices, systems and methods have been described herein. As noted elsewhere, these embodiments have been described for illustrative purposes only and are not limiting. Other embodiments are possible and are covered by the disclosure, which will be apparent from the teachings contained herein. Thus, the breadth and scope of the disclosure should not be limited by any of the above-described embodiments but should be defined only in accordance with claims supported by the present disclosure and their equivalents. Moreover, embodiments or the subject disclosure may include methods, systems and devices which may further include any and l elements from any other disclosed methods, systems, and devices, including any and all elements corresponding to molecular processing. In other words, elements from one or another disclosed embodiments rosy be interchangeable with elements from other disclosed embodiments. In addition, one or more features/elements of disclosed embodiments may be removed and still result in patentable subject matter (and thus, resulting in yet more embodiments of the subject disclosure). Correspondingly, some embodiments of the present disclosure may be patent ably distinct from one and/or another reference/prior art by specifically lacking one or more elements/features of a system, device and/or method disclosed in such prior art. In other words, claims to certain embodiments may contain negative limitation to specifically exclude one or more elements/features resulting in embodiments which are patentably distinct from the prior art which include such features/elements.

1. A molecule retention cassette for retaining tnolecu1es during electrophoresis, the cassette comprising:
a housing;
a lane configured within the housing, the lane having a first elongate edge and a second elongate edge;
an elution module configured to be received in the lane to divide the lane into a first chamber and a second chamber,
a first buffer reservoir positioned adjacent the first elongate edge; and
a second buffer reservoir positioned adjacent the second elongate edge;
wherein:
a first side of the elution module facing the first chamber comprises a porous sterile filtration membrane; and
a second side of the elution module facing the second chamber comprises an ultrafiltration membrane, the ultrafiltration membrane having a pore size to retain molecules during electrophoresis.

2. The cassette of claim 1, further comprising at least one electrode configured within the first chamber and at least one electrode configured within the second chamber.

3. The cassette of claim 1, wherein the ultrafiltration membrane is a 15 kDa ultratilt.

4. The cassette of claim 1, wherein the elution module is centrally positioned between the first buffer reservoir and the second buffer reservoir.

5. The cassette of claim 1, wherein the elution module is positioned between the first buffer reservoir and the second buffer reservoir.

6. The cassette of claim 1, further comprising agarose gel.

7. The cassette of claim 6, wherein the agarose gel is cast next to the porous sterile filtration membrane.

8. The cassette of claim 7, wherein the agarose gel is cast to form a gel column, wherein dimensions of the gel column are configured to minimize loss of target molecules into the first chamber.

9. The cassette of claim 1, wherein the pore size of the ultrafitration membrane is configured to retain DNA during electrophoresis.

10. The cassette of claim 1, wherein the elution module comprises the elution module and the sample well.

11. The cassette of claim 1, wherein the elution module is configured to receive a sample.

12. A molecule retention cassette for retaining molecules during electrophoresis, the cassette comprising:
a housing;
a plurality of lanes configured within a the housing, the plurality of lanes each having a first elongate edge and a second elongate edge;
a plurality of elution modules, wherein each elution module of the plurality of elution modules is configured to be received in each lane of the plurality of lanes to divide each lane into a first chamber and a second chamber;
a first buffer reservoir positioned adjacent the first elongate edge of each lane; and
a second buffer reservoir positioned adjacent the second elongate edge of each lane;
wherein:
a first side of the elution module facing the first chamber of each lane comprises a porous sterile filtration membrane; and
a second side of the elution module facing the second chamber of each lane comprises an ultrafiltration membrane, the ultrafiltration membrane having a pore size to retain molecules during electrophoresis.

13. A method for isolating and collecting target segments of target particles, the method comprising:
receiving a sample in a sample well of an elution module;
receiving an SDS-containing lysis buffer in a first buffer chamber, buffer chamber being configured along a first side of the elution module;
applying a first electrophoresis voltage t grate components of e sample towards a second buffer chamber configured along a second side of the elution module, such that:
target particles are immobilized in a gel segment configured along the second side of the elution module between the elution module and the second buffer chamber, and
non-target particles pass through the gel segment and into the second buffer chamber;
washing the first buffer chamber, the second buffer chamber, and the elution module;
filling the first buffer chamber, the second buffer charmer, and the elution module with a Cas9 reaction buffer;
emptying the elution module;
refilling the elution module with a Cas9 enzyme mix to cleave sections of target particles immobilized in the gel segment;
loading the elution module with an SDS stop solution;
applying a second electrophoresis voltage to release the Cas9 froth the target particles and migrate Cas9 into the second buffer chamber;
washing the first buffer chamber, the second buffer chamber, and the elution module;
filling the first buffer chamber, the second buffer chamber, and th e elution module with elution buffer; and
applying a third electrophoresis voltage in a reverse direction to migrate the cleaved sections of the target particles from the gel segment and into the elution module.

14. The method of claim 13, wherein the lost side of the elution module comprises an ultrafilter and the second side of the elution module comprises a porous sterile filter.

15. The method of claim 14, wherein the porous sterile filter prevents the cleaved sections of the target particles from leaving the elution module during and after the application of the third electrophoresis voltage.

16. The method of claim 13, wherein the target particles are DNA and wherein the cleaved sections of the DNA comprise desired genomic targets.

17. The method of claim 13, wherein tale application of the second electrophoresis voltage is shorter than the application of the first electrophoresis voltage.

18. The method of claim 13 or 17, wherein the application of the second electrophoresis voltage is shorter than the application of the third electrophoresis voltage.

19. The method of claim 13, wherein application of the first electrophoresis voltage migrates SDS through the elution module n the sample and coat the non-target particles of the sample such that the non-target particles pass through the gel segment and into the second buffer chamber.

20. The method of claim 13, wherein application of the second electrophoresis voltage migrates particles smaller than the target particles into the second buffer chamber.

21. Art electrophoretic instrument system, the system comprising
an, electrophoresis station;
a drawer configured to receive at least one electrophoresis cassette if claim 1 or claim 12;
a liquid handling robot; and
a lateral extension arm configured to moves the drawer laterally, wherein
moving the drawer in a first lateral direction exposes a first side of the at least one cassette to the liquid handling robot, and
moving the drawer in a second lateral direction inserts the drawer into the electrophoresis station;
wherein electrophoresis section houses electrodes corresponding to the at least one cassette, the electrodes configured to apply electrophoresis voltages.

22. The system of claim 21, farther comprising at least one cold storage compartment and at least one room temperature storage compartment.
